(19) United States
(12) Patent Application Publication
(10) Pub. No.: US 2005/0085773 A1
(43) Pub. Date: Apr. 21, 2005

(54) Method and apparatus for locating vascular punctures

(76) Inventor: Andrew Thomas Forsberg, Minneapolis, MN (US)

Correspondence Address: L. Grant Foster;HOLLAND & HART LLP, 555 - 17th Street, Suite 3200, P.O. Box 8749, Denver, CO, 80201, US

(21) Appl. No.: 10/686,077

(22) Filed: Oct. 15, 2003

(51) Int. Cl. A61M 5/178

(52) U.S. Cl. 604/164.01; 606/213

(57) ABSTRACT

The present invention provides vascular insertion sheaths and other tools having penetration locators. The insertion sheaths or other tools also have a switch for controlling fluid flow therethrough. The switch may be a valve or a closed indication chamber that reduces or eliminates blood loss to atmosphere while still providing a visual indication of lumen penetration.

### FIELD OF THE INVENTION

The present invention relates to medical devices, and, more particularly, to a vascular insertion device with a closed or controlled blood flow path.

### BACKGROUND OF THE INVENTION

Various medical procedures, particularly cardiology procedures, involve accessing a corporeal vessel or other lumen through a percutaneous sheath. The sheath necessarily requires the formation of a hole or opening in the vessel wall so that a medical procedure can be performed via the sheath. After the particular medical procedure has been performed, the sheath must eventually be removed from the vessel and the access hole in the vessel wall must be closed.

A number of prior vascular closure devices have been developed to close the vessel wall. Closing the vessel wall typically involves packing a resorbable sealing plug at the hole or sandwiching the hole between the sealing plug and an anchor. Examples of prior vascular closure devices are described in U.S. Pat. Nos. 6,179,863; 6,090,130; and 6,045,569 and related patents that are hereby incorporated by reference.

However, prior to a successful deployment of the sealing plug or another vascular tool, an insertion sheath must be properly located within the vessel or other lumen. Proper placement of the insertion sheath enables proper placement of the sealing plug or insertion of a vascular tool.

According to conventional techniques, proper placement of the insertion sheath is accomplished with the aid of a puncture locator. Typically a puncture locator and insertion sheath are inserted through the hole in the vessel wall. The puncture locator provides a fluid communication path from a distal tip (where the insertion sheath enters the vessel) to a proximal end, where blood flow can be observed by an operator. As the sheath penetrates the vessel wall, blood flows through and out of the puncture locator. Blood exiting the puncture locator indicates that the insertion sheath has begun to penetrate the blood vessel. Blood will continue to flow through the puncture locator until the sheath and/or puncture locator are removed from the vessel.

While the flow of blood is important to finding the vessel, blood often flows regularly or continuously through the puncture locator during some vascular sealing procedures. A continuous flow of blood exposes heath care providers to the blood and can result in significant blood loss to the patient. Thus, it is desirable to provide an improved vascular penetration depth locator.

### SUMMARY OF THE INVENTION

In one of many possible embodiments, the present invention provides a vascular insertion apparatus, comprising a vascular insertion sheath, a tissue puncture sealing device, and a switch disposed along a fluid communication path in the vascular insertion sheath, the tissue puncture sealing device, or both, for controlling blood flow from a subcutaneous puncture. The switch may be a visual indication chamber, a valve such as a push button valve, or some other apparatus. According to embodiments where the switch is a visual indication chamber, the visual indication chamber may include at least one valve for adjusting pressure inside the visual indication chamber. The valve of the visual indication chamber may be a luer valve.

According to some embodiments the switch controls blood flow from multiple streams or lumens. Accordingly, the switch may be a multiple luer valve, one for each of the multiple streams or lumens.

According to another aspect of the invention, there is a vascular insertion apparatus including a vascular puncture locator, an insertion sheath receptive of the puncture locator, a fluid communication path through the puncture locator, and a valve disposed along the fluid communication path for selectively opening and closing the fluid communication path through the puncture locator on demand. The valve according to some embodiments is a push button valve. The push button valve may include a mandrel with at least one flow passage therethrough, where the at least one flow passage is open to the fluid communication path in an open position and is closed to the fluid communication path in a closed position. The mandrel may further comprise a stem extending through a hole in the puncture locator and a depressible button at an end of the stem. In addition, a biasing member such as a spring may be disposed between the depressible button and the puncture locator to bias the push button valve in either the open or closed position. According to some aspect of this embodiment, the puncture locator may further include at least two fluid communication paths, and the push button valve or at least one additional valve may be included to control the additional fluid communication path through the puncture locator.

Another embodiment of the present invention provides a vascular apparatus including a vascular insertion sheath having an internal passageway for allowing the passage of vascular instruments or closure devices therethrough, the vascular insertion sheath including a fluid communication path separate from the internal passageway, and a switch along a fluid communication path through the sheath for controlling blood flow from a subcutaneous puncture. The switch may be a closed visual indication chamber and may comprise at least one luer valve for adjusting pressure inside the visual indication chamber. The switch may also be a push button valve.

Another embodiment provides a vascular insertion assembly including an insertion sheath having a distal end, a proximal end, and an inside diameter; a puncture locator sized to fit in the inside diameter of the insertion sheath, the puncture locator having a distal end and a proximal end; a first inlet port located at the distal end of the sheath; a first closed visual indicator in fluid communication with the first inlet port, such that when the first inlet port penetrates a vessel, the first indicator provides visual indication without discharging blood to atmosphere. The assembly may also include a second inlet port located at the distal end of the puncture locator, and a second closed visual indicator in fluid communication with the second inlet port, such that when the second inlet port penetrates a vessel the second indicator provides indication without discharging blood to atmosphere. The first closed visual indicator may be a first drip hole in fluid communication with the first inlet port and enclosed by a chamber. Similarly the second indicator may be a second drip hole in fluid communication with the second inlet port and enclosed by the same or another chamber.

Another embodiment provides a tissue puncture closure device for partial insertion into and sealing of an internal tissue wall puncture including a filament extending from a first end of the closure device to a second end of the closure device, an anchor for insertion through the tissue wall puncture attached to the filament at the second end of the closure device, a sealing plug slidingly attached to the filament adjacent to the anchor, and a switch for controlling blood flow from the tissue wall puncture through the tissue closure device. The switch may include a closed visual indication chamber, a push button valve, or another device.

Another aspect of the invention provides a method of controlling blood flow from a vascular insertion apparatus including providing a vascular insertion sheath having a fluid communication path therethrough, providing a switch disposed along a fluid communication path in the vascular insertion sheath that is selectively operated to permit or restrict blood flow or pulsations from a subcutaneous puncture through the fluid communication path in the vascular insertion sheath.

Another aspect of the invention provides a method of locating a vascular puncture including inserting a vascular instrument into a percutaneous incision, allowing a flow of blood to enter a fluid passageway disposed in the vascular instrument, controlling the flow of blood allowed to enter the fluid passageway, and monitoring a visual indicator of blood flow. The controlling may include opening or closing a valve disposed along the fluid passageway, and the monitoring may include looking for a blood spurt exiting the fluid passageway. The controlling may alternatively include adjusting pressure within a closed chamber in fluid communication with the fluid passageway, and the monitoring may include looking for blood pulsation in the closed chamber.

The foregoing and other features, utilities and advantages of the invention will be apparent from the following more particular description of preferred embodiments of the invention as illustrated in the accompanying drawings.

### BRIEF DESCRIPTION OF THE DRAWING

The accompanying drawings illustrate various embodiments of the present invention and are a part of the specification. The illustrated embodiments are merely examples of the present invention and do not limit the scope of the invention.

FIG. 1 is a perspective view of an insertion sheath and puncture locator with a flow control switch shown in a first position according to one embodiment of the present invention.

FIG. 2 is a perspective view of the insertion sheath and puncture locator of FIG. 1 in a second position with the puncture locator extending into a lumen.

FIG. 3 is a detailed perspective view of an insertion sheath and an insertion instrument with multiple fluid passages according to another embodiment of the present invention.

FIG. 4A is an end view of a push button valve according to one embodiment of the present invention.

FIG. 4B is a cross-sectional side view of the push button valve of FIG. 4A, shown in a first position.

FIG. 4C is a cross-sectional side view of the push button valve of FIG. 4A, shown in a second position.

FIG. 5A is a cross-sectional side view of a push button valve shown in a first position according to one embodiment of the present invention.

FIG. 5B is a cross-sectional side view of the push button valve of FIG. 5A and shown in a second position.

FIG. 6A is a cross-sectional side view of a cam and lever switch shown in a first position according to one embodiment of the present invention.

FIG. 6B is a cross-sectional side view of the cam and lever switch of FIG. 6A, shown in a second position.

FIG. 7A is a side view of a chamber and valves shown with the valves in a first position according to one embodiment of the present invention.

FIG. 7B is a side view of the chamber of FIG. 7A shown with the valves in a second position.

FIG. 8 is a perspective view of an insertion sheath and puncture locator with a flow control switch on the insertion sheath according to one embodiment of the present invention.

FIG. 9 is a perspective view of an insertion sheath and puncture locator with a flow control switch shown according to another embodiment of the present invention.

FIG. 10 a perspective view of an insertion sheath and puncture closure device having a flow control switch according to another embodiment of the present invention.

FIG. 11 a perspective view of an insertion sheath and puncture closure device having a flow control switch according to another embodiment of the present invention

Throughout the drawings, identical reference numbers designate similar, but not necessarily identical, elements.

### DETAILED DESCRIPTION

As mentioned above, vascular procedures are commonly performed throughout the world and require access to a lumen through a puncture. Often an insertion sheath is placed in the puncture to facilitate access to the lumen by one or more vascular instruments, including puncture closure devices. Typically the location of an artery or other lumen is indicated by a flow of blood through a vascular instrument as the instrument enters the artery. The present invention describes methods and apparatus to control fluid flow from various vascular instruments used to locate a lumen. While the vascular instruments shown and described below include insertion sheaths, puncture locators, and puncture sealing devices, the application of vascular flow control is not limited to these specific devices. The principles described herein may be used to control fluid flow for any vascular device, particularly vascular devices used to locate an artery. Therefore, while the description below is directed primarily to arterial procedures and the fluid referenced most often is blood flowing through an artery, the method and apparatus may be used according to principles described herein with any lumen to control the flow of any fluid.

As used throughout the claims and specification the term “switch” is used broadly to encompass any device used to control. The term “fluid” refers to any substance whose molecules move freely past one another and that has the tendency to assume the shape of its container, including both liquids and gasses. A “lumen” refers to any open space or cavity in a bodily organ, especially in a blood vessel.

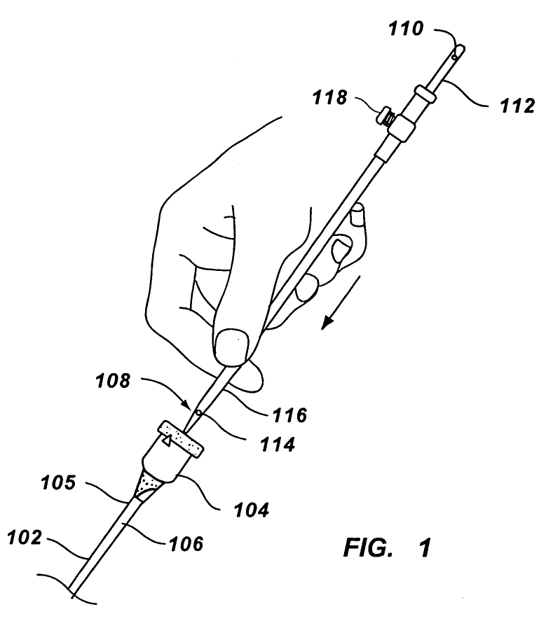

Referring now to the drawings, and in particular to FIG. 1, a vascular insertion apparatus 100 according to one embodiment of the present invention is shown. According to the embodiment of FIG. 1, the vascular insertion apparatus 100 includes a vascular insertion sheath 102 with a funnel 104 disposed at a proximal end 105 thereof. The vascular insertion sheath 102 provides access to an artery or other lumen via an inside diameter 106 that is receptive of a puncture locator 108 or other vascular instrument.

The puncture locator 108 is often used to locate an artery during various vascular procedures. For example, the puncture locator 108 is used to locate an artery after a vascular procedure had been completed and the puncture in the artery is to be sealed. The puncture locator 108 is inserted through the insertion sheath 102 and into a bodily lumen until a flow of blood is observed exiting a drip hole 110 located at a proximal end 112 of the puncture locator 108. When the puncture locator 108 enters an artery, blood flowing through the artery enters through a first port 114 disposed at a distal end 116 of the puncture locator 108. The first port 114 is in fluid communication with the drip hole 110 via a fluid communication path extending through the puncture locator 108. The drip hole 110 is the exit point for the fluid communication path through the puncture locator. Normally, when the puncture locator 108 enters the artery, blood spurts from the drip hole 110 in a pattern corresponding to a patient's heartbeat. However, according to conventional methods and apparatus, the release of blood to atmosphere through the drip hole 110 is uncontrolled. As long as the puncture locator (or other instrument) is inserted into the artery (and sufficient blood pressure exists), blood flows or spurts through the drip hole 110.

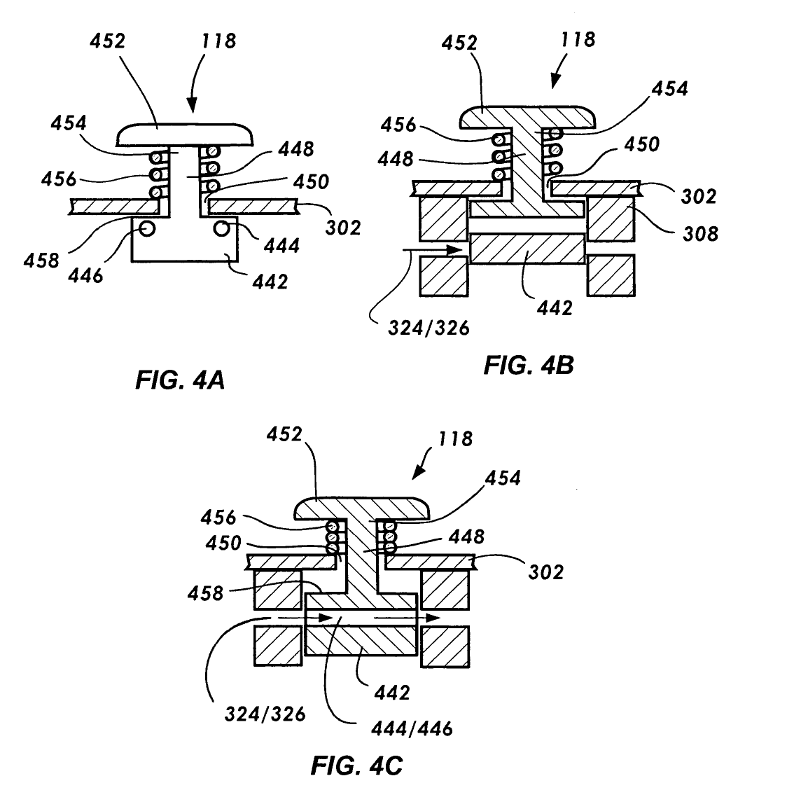

Therefore, according to one embodiment of the present invention, there is a switch disposed along the fluid communications path through the puncture locator 108 to control or eliminate the release of blood to atmosphere, while still visually confirming location of the insertion sheath 102, puncture locator 108, or other vascular instrument within the artery. According to the embodiment of FIG. 1, the switch is a push-button valve 118 located at the proximal end 112 of the puncture locator 108. The push-button valve 118 controls fluid flow through the fluid communication path in the puncture locator 108 when the puncture locator is inserted into an artery. The push-button valve 118 may be normally open or normally closed, preferably normally closed. Details of some embodiments of the push button valve 118 are discussed in more detail below with reference to FIGS. 4A-6B.

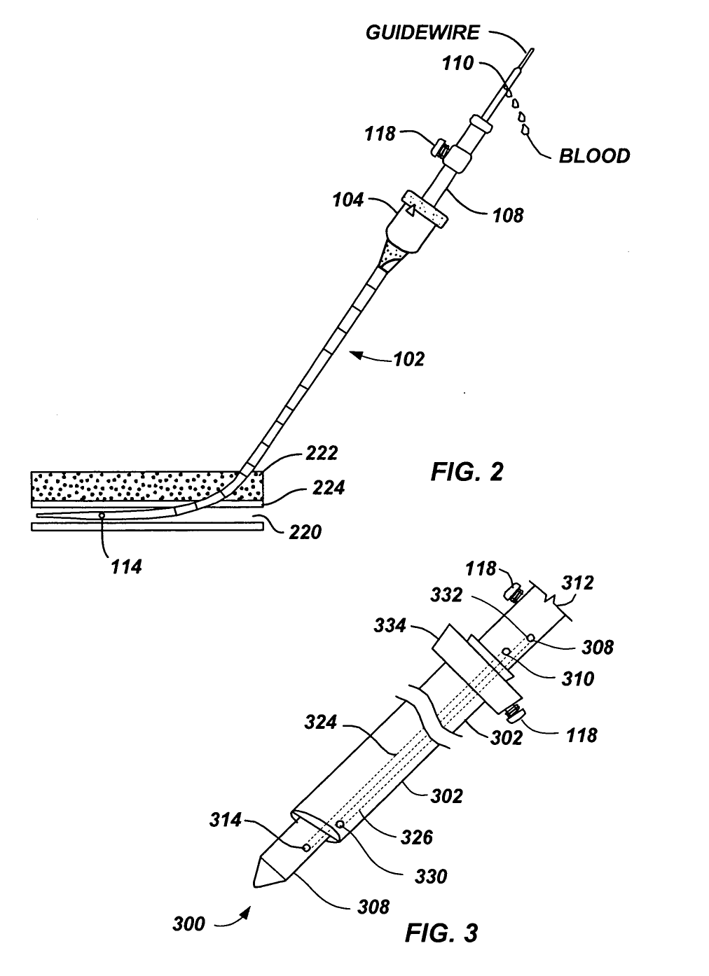

According to the embodiment of FIG. 1, fluid is allowed to flow through the fluid communication path of the puncture locator 108 and exit the drip hole 110 only when the push button valve 118 is depressed and open. Therefore, as shown in FIG. 2, with the insertion sheath 102 engaging a subcutaneous incision 222, an operator may depress the push-button valve 118 and insert the puncture locator 108 through the insertion sheath 102 and into an artery 220. As the puncture locator 108 enters the artery 220, a volume of blood enters the fluid communication path through the first port 114. If the push-button valve 118 continues to be depressed as shown, the fluid communication path is open to the drip hole 110. Accordingly, an operator will observe a flow or spurt of blood from the drip hole 112 when the puncture locator 108 enters the artery 220.

However, whereas prior devices allow the flow of blood from the drip hole 110 to continue in an uncontrolled manner, the present invention provides a control mechanism. According to the present embodiment of the invention shown in FIGS. 1-2, as soon as the operator is satisfied that the artery 220 has been located, the push-button valve 118 is released, which stops the flow of blood through the communication path of the puncture locator 108. Therefore, only an insubstantial volume of blood is released to atmosphere, reducing the exposure of healthcare professionals to the discharged blood, and minimizing fluid loss to the patient.

Referring next to FIG. 3, another embodiment of a vascular insertion apparatus 300 is shown. According to the embodiment of FIG. 3, there is an insertion sheath 302 and a puncture locator 308 that comprise multiple fluid communication paths, for example a first fluid communication path 324 through the puncture locator 308 and a second communication path 326 through the insertion sheath 302. The first fluid communication path 324 extends from a first inlet port 314 in the puncture locator 308 to a first drip hole 310 located at a proximal end 312 of the puncture locator 308. Similarly, the second fluid communication path 326 extends from a second inlet port 330 in the insertion sheath 302 to a second drip hole 332 disposed at a proximal end 334 of the insertion sheath 302. According to this embodiment, one or more push-button valves 118 or other switches control the flow through one or both of the fluid communication paths 324, 326 in a manner similar or identical to that described above with reference to FIG. 1. However, the use of multiple fluid communication paths, each having a different location, may provide an operator with additional location information. For example, flow from the first drip hole 310 may indicate entry into an artery, while flow from the second drip hole 332 may indicate over-insertion into the artery. And while the present embodiment illustrates two fluid communication paths 324, 326, there may any number of paths used, with one or more of the paths including a switch for controlling flow according to the present invention.

The push button valve of FIGS. 1-3 118 may include any convenient configuration for opening and closing flow through a fluid communication path. One embodiment of the push-button valve 118 according to the present invention is illustrated in FIGS. 4A-4C. FIG. 4A is a front view of the push-button valve 118. According to the embodiment of FIG. 4A, the push-button valve 118 includes a mandrel 442 with at least one flow passage 444 extending therethrough. According to the embodiment of FIG. 4A, however, there are two flow passages 444, 446 extending through the mandrel 442, providing for flow control for multiple fluid communication paths. Additional flow passages may also be included in some embodiments of the push-button valve 118.

The flow passages 444, 446 may, for example, correspond to the fluid communication paths 324, 326 of FIG. 3, respectively. The flow passages 444, 446 are closed to the fluid communication paths 324,326 (FIG. 3) found in the puncture locator 308 and insertion sheath 302 in a closed position, as shown in FIG. 4B. According to FIG. 4B, the flow passages 444, 446 are misaligned with the fluid communication paths 324, 326 and thus the push-button valve 118 prevents the passage of fluid therethrough.

However, when the push-button valve 118 is depressed, as shown in FIG. 4C, the flow passages 444, 446 are aligned with and open to the fluid communication paths 324, 326 in the insertion sheath 302 and the puncture locator 308. In addition, the push-button valve 118 may be integrated with any other vascular location tool having a fluid flow passageway therethrough and is not limited to the insertion sheath 302/puncture locator 308 assembly shown in FIG. 3.

As also shown in FIGS. 4A-4C, the push-button valve 118 has a stem 448 extending through a hole 450 in the insertion sheath 302, and includes a depressible button 452 at an end 454 of the stem 448. In addition, a biasing member 456 may be disposed between the depressible button 452 and the insertion sheath 302 or other tool. The mandrel 442 further includes a shoulder 458 opposite of the depressible button 452 and interior to the insertion sheath 302 such that the shoulder 458 is larger than the hole 450 in the insertion sheath 302.

According to the embodiment of FIGS. 4A-4C, the combination of the depressible button 452, the stem 448, and the shoulder 458, comprise a generally I-shape. The push-button valve 118 is normally biased toward the closed position in the embodiment shown in FIG. 4B. However, a normally closed bias is not a requirement, as the push-button valve 118 may also be biased to an open position. Further, while the embodiment of FIGS. 4A-4C illustrate the push-button valve extending through both the insertion sheath 302 and the puncture locator 308 to control both the first and second fluid communication passageways 324, 326, the push-button valve 118 may be added separately and independently to the insertion sheath 302 and the puncture locator 308 as shown in FIG. 3.

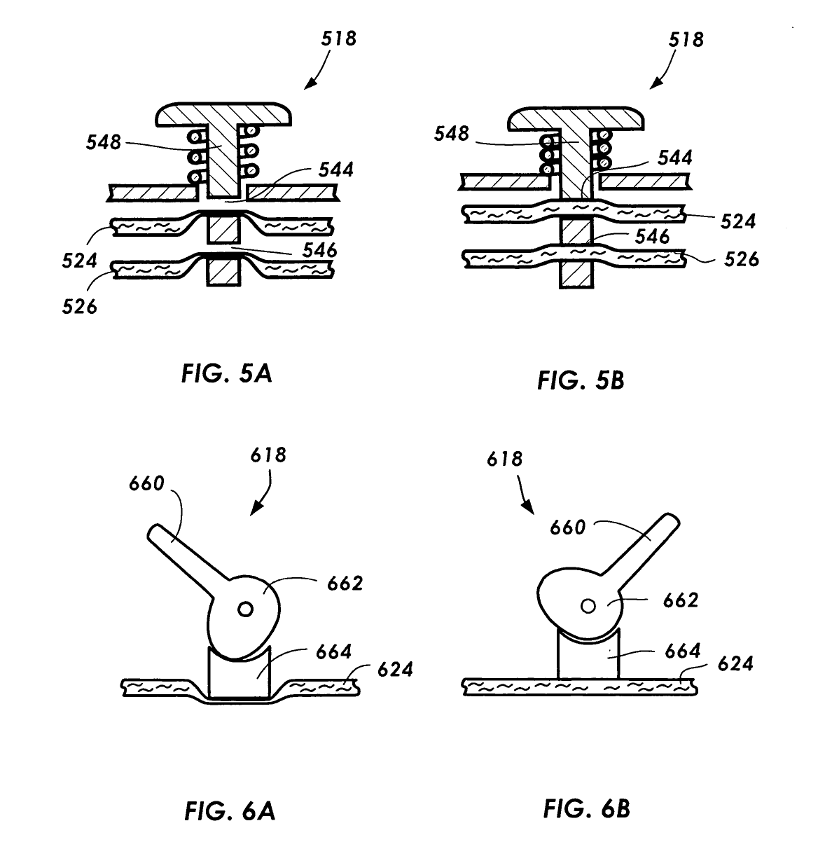

Referring next to FIGS. 5A-5B, an alternative embodiment of a push-button valve 518 is shown. According to the alternative embodiment of FIGS. 5A-5B, there is at least one flow passage 544 disposed in a stem 548 of the push-button valve 518. It will be understood by those of skill in the art having the benefit of this disclosure, however, that additional flow passages, such as the second passage 546 shown in FIGS. 5A and 5B, may also be used. According to the embodiment of FIGS. 5A and 5B, instead of aligning the flow passages 544, 546 with fluid communication paths through associated vascular insertion devices, the stem 548 allows one or more fluid communication tubes 524, 526 to extend therethrough. In a first or closed position shown in FIG. 5A, the fluid communication tubes 524, 526 are closed, crimped by the stem 548 to prevent flow therethrough. When it is desired by an operator to have visual indication of insertion of a vascular location device in an artery or other lumen, the push-button valve 518 is depressed as shown in FIG. 5B, in a similar manner as described previously with reference to FIG. 4C. As the push-button valve 518 is depressed, the flow communication tubes 524, 526 are no longer crimped by the stem 548, and fluid may freely pass through. Fluid passing through the flow communication tubes 524, 526 may exit a drip hole such as the one shown in FIGS. 1-2 (drip hole 110), where it is observed by an operator or other healthcare professional to locate the artery or other lumen. Again, as soon as the operator is satisfied with the placement of an insertion device (such as the insertion sleeve 302 or the puncture locator 308 (FIG. 3)), the push-button valve 518 may be released and the flow of blood or other fluid through the flow communication tubes 524, 526 ceases.

Referring next to FIGS. 6A-6B, yet another switch for controlling fluid flow is shown according to one embodiment of the present invention. Referring first to FIG. 6A, the switch is a valve 618 comprising a lever 660 operatively connected to a cam 662. According to the embodiment of FIG. 6A-6B, the cam 662 and lever 660 are integrally formed. The valve 618 is shown in a closed position in FIG. 6A wherein the cam 662 engages an insert 664, which is shown to be crimping or closing a fluid communication path or tube 624 extending through a vascular location device such as the puncture locator 308 (FIG. 3). However, according to some embodiments there is no insert 664 and the cam directly crimps the fluid communication path or tube 624.

When fluid flow through the fluid communication path or tube 624 is desired, the lever 660 may be switched to an open position as shown in FIG. 6B. Moving the lever 660 causes the cam 662 to rotate and release the insert 664 from crimping the fluid flow path 624, thereby allowing fluid flow through the fluid communication path 624. Again, the flow of fluid may then be observed by an operator as an indication of artery location. When the operator is satisfied with device position, he or she may switch the valve 618 back to the closed position (FIG. 6A) to minimize blood loss.

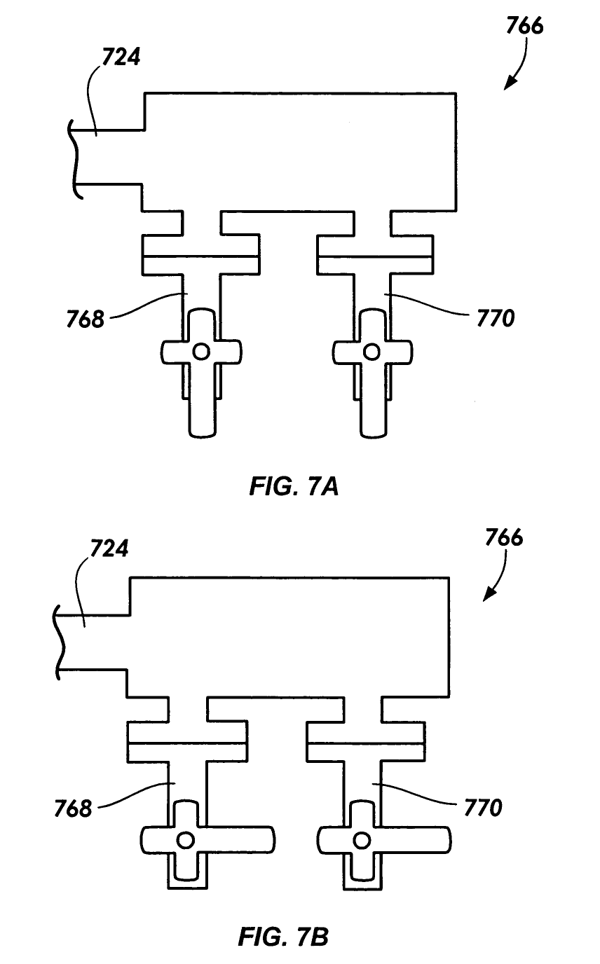

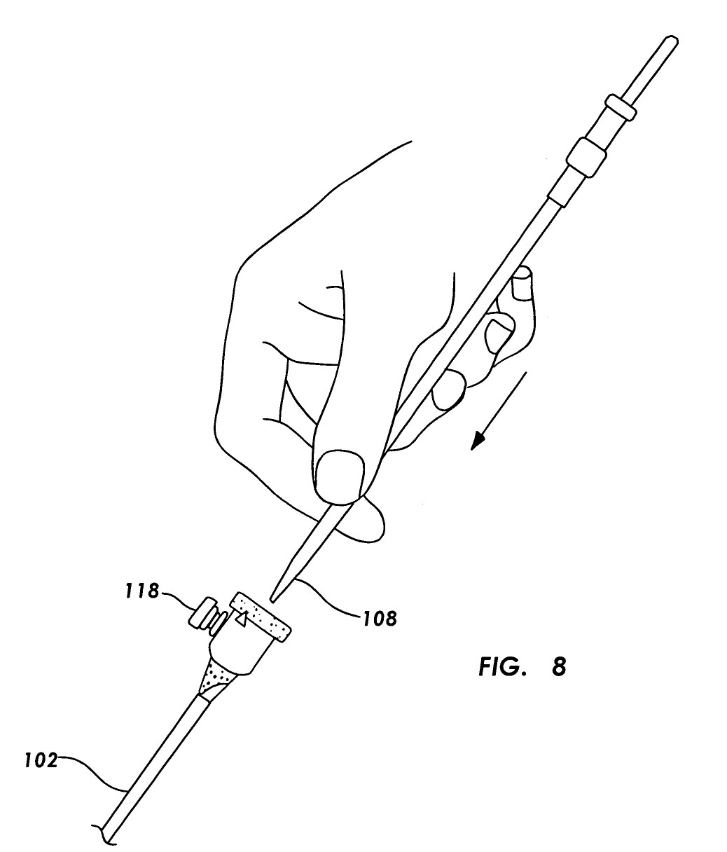

Referring next to FIGS. 7A-7B, a switch according to another embodiment of the present invention is shown for controlling fluid flow through a vascular device. According to the embodiment of FIGS. 7A-7B, the switch comprises a visual indication chamber 766. The visual indication chamber 766 is in fluid communication with a fluid flow path 724 of a vascular device such as the puncture locator 308 (FIG. 3), the insertion sheath 302 (FIG. 3), or another device. The visual indication chamber 766, however, is closed and does not normally allow fluid passage all the way therethrough. Accordingly, the visual indication chamber 766 provides indication of artery location with little or no blood loss.

Preferably, the visual indication chamber 766 is transparent or translucent. Therefore, as an insertion device enters an artery, blood will flow into the visual indication chamber 766 via the fluid flow path 724. To encourage the flow of blood into the visual indication chamber 766 via the fluid flow path 724, the visual indication chamber 766 may include at least one valve 768, and, according to the embodiment of FIGS. 7A and 7B, there are two valves 768, 770. The valves 768, 770 may comprise luer valves that are widely available from a variety of sources. The valves 768, 770 may be opened to vent air or other fluids that may be present in the visual indication chamber 766 at a pressure that reduces or prevents blood flow into the visual indication chamber 766. Therefore, as an insertion device including the visual indication chamber 766 is inserted into a lumen, one or more of the two valve 768, 770 may be in a first or open position as shown in FIG. 7A. When the instrument penetrates the lumen and a flow of blood or other fluid enters the visual indication chamber 766, the valves 768, 770 may be closed as shown in FIG. 7B, preventing the release of blood to atmosphere. The visual indication chamber 766 may continue to be monitored for pulsation (corresponding to the beating of a patient's heart) adding confidence to the operator of the location of the vascular location device or other instrument within an artery. Therefore, an operator may have clear and continuing visual indication of proper location of a vascular device within an artery with little or no blood loss. In an alternative embodiment, the visual indication chamber 766 does not include any valves 768, 770.

The switches, such as the visual indication chamber 766 and the push-button switch 118, may be used in a variety of ways with various vascular insertion instruments. For example, the switches may be used with the embodiments described above, including the embodiment of FIG. 3 illustrating multiple fluid steams 324 and 326. A single switch may be used to control all the fluid streams, or each fluid stream may have its own switch.

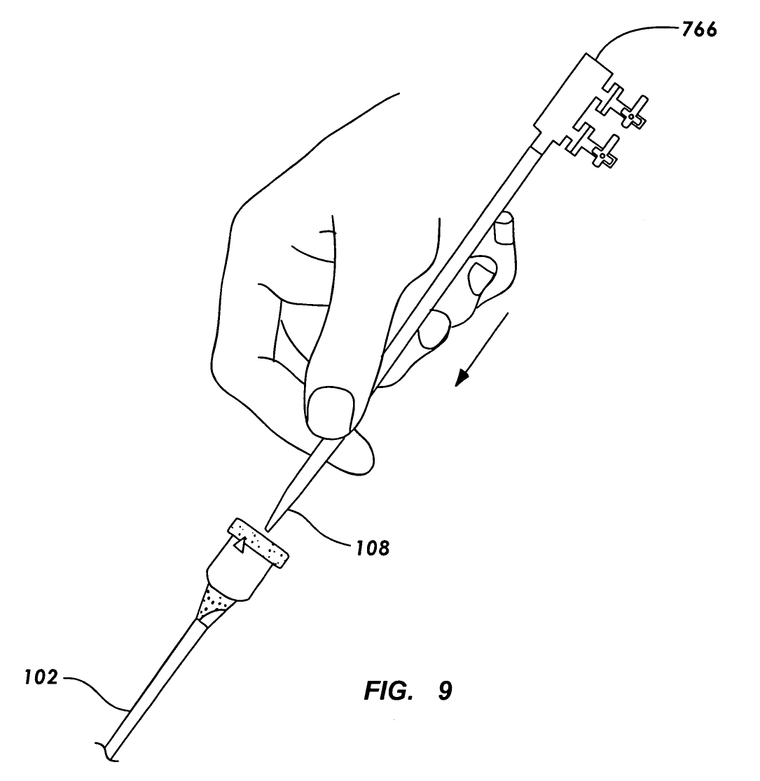

In addition, the switches described above and others may be used according to other embodiments. For example, with reference to FIG. 8, the push-button valve 118 (or other valves), may be added to the insertion sheath 102, as shown. Likewise, with reference to FIG. 9, the visual indication chamber 766 may be coupled to the puncture locator 108.

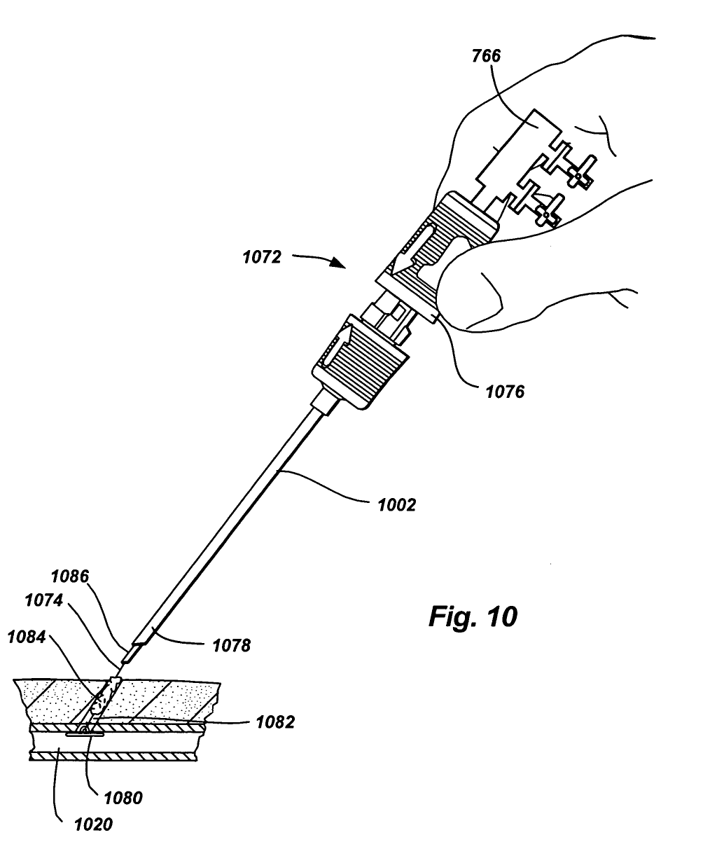

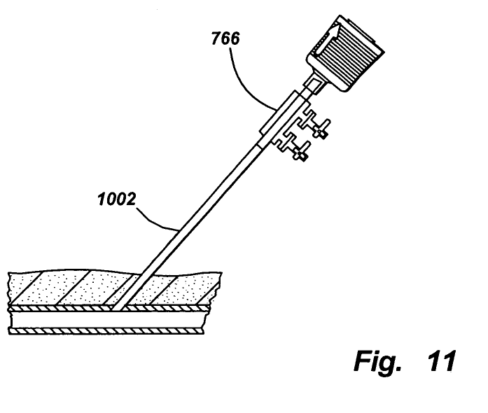

The visual indication chamber 766, or another switch such as the push-button switch 118, may also be used with a puncture closure assembly 1072, as shown in FIG. 10. The puncture closure assembly 1072 must be precisely located in the artery 1020 following the completion of a vascular procedure to ensure proper function of the puncture closure assembly 1072. Therefore, according to the embodiments of FIGS. 10-11, the visual indication chamber 766 is integrated with either the puncture closure device 1072 (FIG. 10) or the insertion sheath 1102 (FIG. 11). The closure assembly 1072 may then be inserted through an insertion sheath 1002, while the operator watches the visual indication chamber 766 for blood flow. As mentioned above, blood flow into the visual indication chamber 766 may be aided by opening one or more of the valves 768, 770 to adjust pressure and/or release any air trapped in the chamber 766 or the fluid communication path leading thereto. As blood enters the visual indication chamber 766, the operator knows that the closure assembly 1072 has penetrated the artery 1020, and a seal 1084 may be deployed.

However, according to some embodiments, instead of the visual indication chamber 766, the insertion sheath 1002 or the puncture closure assembly may include the push-button valve 118 (FIG. 1) (or any other valve). The push-button valve 118 supplement or replace the visual indication chamber 766 to control blood flow during and following artery locating.

As shown in FIG. 10, in order to facilitate deployment of the seal 1084, the puncture closure device 1072 may include a filament 1074 extending from a first or proximal end 1076 of the closure device 1072 to a second or distal end 1078 of the closure device 1072. The puncture closure device 1072 also includes an anchor 1080 for insertion through a tissue wall puncture 1082, through which the filament 1074 is threaded at the second end 1078 of the closure device 1072. The sealing plug 1084 is slidingly attached to the filament 1074 adjacent to the anchor 1080. The sealing plug 1084 may be automatically or manually tamped with a tamping tube 1086. Tamping the sealing plug 1084 sandwiches the puncture 1082 between the anchor 1080 and the sealing plug 1084. The filament 1072 may then be tied and cut, and the closure device 1072 may be removed. Accordingly, the puncture 1082 may be located and sealed according to the present invention with little or no blood loss to the patient.

While the invention has been particularly shown and described with reference to embodiments thereof, it will be understood by those skilled in the art that various other changes in the form and details may be made without departing from the scope of the invention.

1. A vascular insertion apparatus, comprising:
a vascular insertion sheath;
a tissue puncture sealing device;
a switch in the vascular insertion sheath, the tissue puncture sealing device, or both for controlling blood flow from a subcutaneous puncture.

2. The vascular insertion apparatus according to claim 1 wherein the switch comprises a visual indication chamber.

3. The vascular insertion apparatus according to claim 2 wherein the visual indication chamber comprises at least one valve for adjusting pressure inside the visual indication chamber.

4. The vascular insertion apparatus according to claim 3 wherein the at least one valve comprises a luer valve.

5. The vascular insertion apparatus according to claim 1 wherein the switch controls blood flow from multiple streams or lumens.

6. The vascular insertion apparatus according to claim 5 wherein the switch comprises one or more luer valves.

7. The vascular insertion apparatus according to claim 1 wherein the switch comprises a push button valve.

8. A vascular insertion apparatus according to claim 7 wherein the push button valve comprises a mandrel with at least one flow passage therethrough, wherein the at least one flow passage is open to the blood flow in an open position and is closed to the blood flow in a closed position.

9. A vascular insertion apparatus according to claim 8 wherein the mandrel further comprises:
a stem extending through a hole in the vascular insertion sheath, the arterial sealing device, or both;
a depressible button at an end of the stem.

10. A vascular insertion apparatus according to claim 9, further comprising a biasing member disposed between the depressible button and the vascular insertion sheath or arterial sealing device, the biasing member biasing the push button valve to the closed position.

11. A vascular insertion apparatus according to claim 9, further comprising at least two flow passages through the push button valve mandrel.

12. A vascular insertion apparatus according to claim 11 wherein the push button valve operates to open or close all of the at least two flow passages through the mandrel.

13. A vascular insertion apparatus according to claim 1 wherein the switch comprises a lever arm and cam.

14. A vascular insertion apparatus, comprising:
a vascular puncture locator;
an insertion sheath receptive of the puncture locator;
a fluid communication path through the puncture locator;
a valve disposed along the fluid communication path for selectively opening and closing the fluid communication path through the puncture locator on demand.

15. A vascular insertion apparatus according to claim 14 wherein the valve comprises a push button valve.

16. A vascular insertion apparatus according to claim 15 wherein the push button valve comprises a mandrel with at least one flow passage therethrough, wherein the at least one flow passage is open to the fluid communication path in an open position and is close to the fluid communication path in a closed position.

17. A vascular insertion apparatus according to claim 16 wherein the mandrel further comprises a stem extending through a hole in the puncture locator and a depressible button at an end of the stem.

18. A vascular insertion apparatus according to claim 17, further comprising a biasing member disposed between the depressible button and the puncture locator.

19. A vascular insertion apparatus according to claim 17 wherein the mandrel further comprises a shoulder disposed opposite of the depressible button and interior to the puncture locator, wherein the shoulder is larger than the hole in the puncture locator.

20. A vascular insertion apparatus according to claim 19 wherein the combination of the depressible button, stem, and shoulder comprises a generally “I” shape.

21. A vascular insertion apparatus according to claim 16, further comprising at least two fluid communication paths through the puncture locator.

22. A vascular insertion apparatus according to claim 21 wherein the push button valve operates to open or close all of the at least two fluid communication paths through the puncture locator.

23. A vascular insertion apparatus according to claim 21 further comprising at least one additional valve for each additional fluid communication path through the puncture locator.

24. A vascular insertion apparatus according to claim 14 wherein the fluid communication path comprises at least one tube and the valve comprises a mandrel with a hole therethrough, the at least one tube extending through the hole.

25. A vascular insertion apparatus according to claim 24 wherein the mandrel crimps the at least one tube in a closed position.

26. A vascular insertion apparatus according to claim 14 wherein the valve comprises a lever and cam.

27. A vascular apparatus, comprising:
a vascular insertion sheath having an internal passageway for allowing the passage of vascular instruments or closure devices therethrough, the vascular insertion sheath comprising a switch along a fluid communication path through the sheath for controlling blood flow from a subcutaneous puncture; wherein the fluid communication path through the sheath is separate from the internal passageway.

28. The vascular apparatus according to claim 27 wherein the switch comprises a visual indication chamber.

29. The vascular insertion apparatus according to claim 28 wherein the visual indication chamber comprises at least one luer valve for adjusting pressure inside the visual indication chamber.

30. The vascular insertion apparatus according to claim 27 wherein the switch controls blood flow from multiple streams or lumens.

31. The vascular insertion apparatus according to claim 27 wherein the switch comprises a push button valve.

32. A vascular insertion assembly, comprising:
an insertion sheath having a distal end, a proximal end, and an inside diameter;
a puncture locator sized to fit in the inside diameter of the insertion sheath, the puncture locator having a distal end and a proximal end;
a first inlet port located at the distal end of the sheath;
a first closed visual indicator in fluid communication with the first inlet port, such that when the first inlet port penetrates a vessel the first indicator provides visual indication without discharging blood to atmosphere.

33. The vascular insertion assembly according to claim 32, further comprising:
a second inlet port located at the puncture locator distal end;
a second closed visual indicator in fluid communication with the second inlet port, such that when the second inlet port penetrates a vessel, the second indicator provides indication without discharging blood to atmosphere.

34. The vascular insertion assembly according to claim 32, wherein the first closed visual indicator is a first drip hole in fluid communication with the first inlet port and enclosed by a chamber.

35. The vascular insertion assembly according to claim 33, wherein the second indicator is a second drip hole in fluid communication with the second inlet port and enclosed by a chamber.

36. A tissue puncture closure device for partial insertion into and sealing of an internal tissue wall puncture, comprising:
a filament extending from a first end of the closure device to a second end of the closure device;
an anchor for insertion through the tissue wall puncture attached to the filament at the second end of the closure device;
a sealing plug slidingly attached to the filament adjacent to the anchor;
a switch for controlling blood flow from the tissue wall puncture through the tissue closure device.

37. The vascular insertion apparatus according to claim 36 wherein the switch comprises a closed visual indication chamber.

38. The vascular insertion apparatus according to claim 37 wherein the visual indication chamber comprises at least one valve for adjusting pressure inside the visual indication chamber.

39. The vascular insertion apparatus according to claim 38 wherein the at least one valve comprises a luer valve.

40. The vascular insertion apparatus according to claim 36 wherein the switch comprises a push button valve.

41. A vascular insertion apparatus according to claim 40 wherein the push button valve comprises a mandrel with at least one flow passage therethrough, wherein the at least one flow passage is open to the blood flow in an open position and is closed to the blood flow in a closed position.

42. A method of controlling blood flow from a vascular insertion apparatus, comprising:
providing a vascular insertion sheath having in internal diameter receptive of a vascular tool and a fluid communication path separate from the internal diameter;
providing a switch disposed along the fluid communication path in the vascular insertion sheath that is selectively operated to permit or restrict blood flow from a subcutaneous puncture through the fluid communication path in the vascular insertion sheath.

43. A method of controlling blood flow from a vascular insertion apparatus according to claim 42 wherein the switch comprises a closed visual indication chamber or a push button valve.

44. The vascular insertion apparatus according to claim 43 wherein the visual indication chamber comprises at least one luer valve for adjusting pressure inside the visual indication chamber.

45. A method of locating a vascular puncture, comprising:
inserting a vascular instrument or insertion sheath into a percutaneous incision;
allowing a flow of blood to enter a fluid passageway disposed in the vascular instrument or insertion sheath;
controlling the flow of blood allowed to enter the fluid passageway;
monitoring a visual indicator of blood flow.

46. A method of locating a vascular puncture according to claim 45 wherein the controlling comprises opening or closing a valve disposed along the fluid passageway.

47. A method of locating a vascular puncture according to claim 46 wherein the monitoring comprises looking for a blood spurt exiting the fluid passageway.

48. A method of locating a vascular puncture according to claim 45 wherein the controlling comprises adjusting pressure within a chamber in fluid communication with the fluid passageway.

49. A method of locating a vascular puncture according to claim 48 wherein the monitoring comprises looking for blood pulsation in the chamber.
